(19) United States
(12) Patent Application Publication
(10) Pub. No.: US 2019/0333628 A1
(43) Pub. Date: Oct. 31, 2019

(54) MEDICAL IMAGING APPARATUS AND METHOD OF CONTROLLING THE SAME

(71) Applicant: SAMSUNG ELECTRONICS CO., LTD., Suwon-si (KR)

(72) Inventors: Hyunhee JO, Suwon-si (KR); Daehwan KIM, Suwon-si (KR); Keumyong OH, Suwon-si (KR)

(73) Assignee: SAMSUNG ELECTRONICS CO., LTD., Suwon-si (KR)

(21) Appl. No.: 16/394,602

(22) Filed: Apr. 25, 2019

(30) Foreign Application Priority Data
Apr. 25, 2018 (KR) 10-2018-0048015

(51) Int. Cl. G16H 30/40; G06T 19/00; G06F 3/048; A61B 5/00

(52) U.S. Cl. CPC G16H 30/40; G06T 19/003; G16H 30/20; A61B 5/7435; G06F 3/048

(57) ABSTRACT

a medical imaging apparatus includes a display, a memory configured to store one or more instructions, and a processor configured to execute the one or more instructions to generate a rendered three-dimensional medical image of a region of interest (ROI) based on medical imaging data, recognize at least one structure of interest (SOI) included in the rendered three-dimensional medical image, generate at least one SOI icon including at least one image showing the at least one SOI, and control the display to display the at least one SOI icon.

### CROSS-REFERENCE TO RELATED APPLICATION(S)

This application is based on and claims priority under 35 U.S.C. § 119 to Korean Patent Application No. 10-2018-0048015, filed on Apr. 25, 2018, in the Korean Intellectual Property Office, the disclosure of which is incorporated by reference herein in its entirety.

### BACKGROUND

### 1. Field

The disclosure relates to medical imaging apparatuses and methods of controlling the same, and more particularly, to medical imaging apparatuses and methods of controlling the same, which are capable of facilitating user manipulation.

### 2. Description of Related Art

Medical imaging apparatuses may be electronic devices capable of generating and processing various types of medical images. In detail, medical imaging apparatuses may be used to acquire images showing an internal structure of an object. The medical imaging apparatuses may capture and/or process images of details of structures, tissue, fluid flow, etc., inside a body and display the images to a user. A user, for example a medical practitioner, may use medical images output from the medical imaging apparatuses to diagnose an examinee's medical condition and diseases. Various analysis applications have been developed to diagnose diseases that occur in an object by using a medical imaging apparatus.

Such analysis applications may include various graphical user interfaces (GUIs) designed to improve user convenience. For example, an analysis application may provide GUI icons that take the shape of a structure of interest (SOI) included in an image of an object for post-processing such as selecting or segmenting the SOI.

For example, GUI icons may include a shape of an SOI schematized based on a two-dimensional (2D) image of the object. However, GUI icons including a shape of a schematized SOI usually do not intuitively match an SOI included in a 2D image of an object. In particular, when a view of an image showing an object is changed or a 2D image of the object is rotated, a GUI icon may be inconsistent with a shape of the corresponding SOI.

### SUMMARY

Provided are medical imaging apparatuses and methods of controlling the medical imaging apparatuses, which are capable of facilitating more convenient manipulation by a user.

Provided are also medical imaging apparatuses for providing icons having shapes that vary according to a shape of a structure of interest (SOI) included in a view or plane of a rendered three-dimensional (3D) medical image and methods of controlling the medical imaging apparatuses.

Provided are also medical imaging apparatuses for providing a user interface that allows a user to easily identify a position of a lesion and methods of controlling the medical imaging apparatuses.

Additional aspects will be set forth in part in the description which follows and, in part, will be apparent from the description, or may be learned by practice of the presented embodiments.

In accordance with an aspect of the disclosure, a medical imaging apparatus includes a display, a memory configured to store one or more instructions, and a processor configured to execute the one or more instructions to generate a rendered three-dimensional medical image of a region of interest (ROI) based on medical imaging data, recognize at least one structure of interest (SOI) included in the rendered three-dimensional medical image, generate at least one SOI icon including at least one image showing the at least one SOI, and control the display to display the at least one SOI icon.

The at least one image may include an image obtained by reducing a portion corresponding to the at least one SOI in the rendered three-dimensional medical image or an image including a figure representing the at least one SOI that is generated based on a result of segmenting the at least one SOI.

The processor may be further configured to execute the one or more instructions to generate the at least one SOI icon in response to an input for generating the rendered three-dimensional medical image.

The processor may be further configured to execute the one or more instructions to, based on receiving an input for changing a first slice of the rendered three-dimensional medical image to a second slice, update the rendered three-dimensional medical image of the ROI being based on the medical imaging data and the second slice, recognize at least one new SOI included in a result of the updating, and update the at least one image to show the at least one new SOI.

The processor may be further configured to execute the one or more instructions to, based on receiving an input for changing a first view of the rendered three-dimensional medical image to a second view, update the rendered three-dimensional medical image of the ROI based on the medical imaging data and the second view, recognize at least one new SOI included in a result of the updating, and update the at least one image to show the at least one new SOI.

The processor may be further configured to execute the one or more instructions to, based on receiving an input for zooming the rendered three-dimensional medical image, update the rendered three-dimensional medical image of the ROI based on the medical imaging data and the input, recognize at least one new SOI included in a result of the updating, and update the at least one image to show the at least one new SOI.

The processor may be further configured to execute the one or more instructions to generate a plurality of rendered three-dimensional medical images respectively corresponding to a plurality of views, and generate the at least one SOI icon corresponding to an image selected by a user from among the plurality of rendered three-dimensional medical images.

The at least one image may be generated based on the medical imaging data and information about medical imaging.

The at least one SOI icon may include an SOI icon representing an SOI including a lesion, and the SOI icon may include an indication that the lesion is present in the SOI corresponding to the SOI icon.

Based on receiving an input of selecting an SOI icon from among the at least one SOI icon, the processor may be further configured to execute the one or more instructions to control the display to display an image showing an SOI corresponding to the selected SOI icon.

In accordance with an aspect of the disclosure, a method of controlling a medical imaging apparatus includes generating a rendered three-dimensional medical image of a region of interest (ROI) based on medical imaging data, recognizing at least one structure of interest (SOI) included in the rendered three-dimensional medical image, generating at least one SOI icon including at least one image showing the at least one SOI, and displaying the at least one SOI icon.

The at least one image may include an image obtained by reducing a portion corresponding to the at least one SOI in the rendered three-dimensional medical image or an image including a figure representing the at least one SOI that is generated on a result of segmenting the at least one SOI.

The generating of the at least one SOI icon comprises generating the at least one SOI icon in response to an input for generating the rendered three-dimensional medical image.

The method may further include, based on receiving an input for changing a first slice of the rendered three-dimensional medical image to a second slice, updating the rendered three-dimensional medical image of the ROI based on the medical imaging data and the second slice, recognizing at least one new SOI included in a result of the updating, and updating the at least one image to show the at least one new SOI.

The method may further include, based on receiving input for changing a first view of the rendered three-dimensional medical image to a second view, updating the rendered three-dimensional medical image of the ROI based on the medical imaging data and the second view, recognizing at least one new SOI included in a result of the updating, and updating the at least one image to show the at least one new SOI.

The method may further include, based on receiving an input for zooming the rendered three-dimensional medical image, updating the rendered three-dimensional medical image of the ROI based on the medical imaging data and the input, recognizing at least one new SOI included in a result of the updating, and updating the at least one image to show the at least one new SOI.

The method may further include generating a plurality of rendered three-dimensional medical images respectively corresponding to a plurality of views, and generating the at least one SOI icon corresponding to an image selected by a user from among the plurality of rendered three-dimensional medical images.

The method may further include acquiring the medical imaging data from the medical imaging apparatus.

The at least one SOI icon may include an SOI icon representing an SOI including a lesion, and the SOI icon may include an indication that the lesion is present in the SOI corresponding to the SOI icon.

In accordance with an aspect of the disclosure, a non-transitory computer-readable medium may be configured to store instructions which, when executed by a processor, cause the processor to perform the methods described herein.

In accordance with an aspect of the disclosure, a method of controlling a medical imaging apparatus includes acquiring medical imaging data including image data and additional information, generating a rendered three-dimensional medical image of a region of interest (ROI) based on the image data, identifying an anatomical structure in the rendered three-dimensional medical image based on the additional information, identifying a plurality of anatomical sub-structures included in the anatomical structure, as a plurality of structures of interest (SOI), generating a plurality of SOI icons including a plurality of images showing the plurality of SOI; and, displaying the plurality of SOI icons.

The method may further include displaying a plurality of second icons including a plurality of figures corresponding to the plurality of images, wherein the plurality of images are generated by segmenting the plurality of SOI..

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a schematic diagram for explaining a medical image generated using a medical imaging application, according to an embodiment;

FIG. 2 is a schematic diagram of a magnetic resonance imaging (MRI) system according to an embodiment;

FIG. 3 is a block diagram of a configuration of a medical imaging apparatus according to an embodiment;

FIG. 4 is a flowchart of a method of controlling a medical imaging apparatus, according to an embodiment;

FIGS. 5A and 5B illustrate icons generated based on at least one structure of interest (SOI);

FIG. 6 is a flowchart of a method of controlling a medical imaging apparatus, according to an embodiment;

FIGS. 7A and 7B illustrate generation of icons corresponding to a cross-section in a medical image based on an input of selecting the cross-section, according to an embodiment;

FIGS. 8A through 8C illustrate at least one SOI icon that changes according to a view of a medical image, according to an embodiment;

FIG. 9 illustrates a screen displayed by a medical imaging apparatus, according to an embodiment; and

FIGS. 10A and 10B illustrate screens displayed by a medical imaging apparatus, according to an embodiment.

### DETAILED DESCRIPTION

The present specification describes principles of the disclosure and sets forth embodiments thereof to clarify the scope of the disclosure and to allow those of ordinary skill in the art to implement the embodiments of the disclosure. The embodiments of the disclosure may have different forms and should not be construed as being limited to the descriptions set forth herein.

Like reference numerals refer to like elements throughout. The present specification does not describe all components in the embodiments of the disclosure, and common knowledge in the art or the same descriptions of the embodiments of the disclosure will be omitted below. The term “part” or “portion” may be implemented using hardware or software, and according to embodiments of the disclosure, one “part” or “portion” may be formed as a single unit or element or include a plurality of units or elements. Expressions such as “at least one of,” when preceding a list of elements, modify the entire list of elements and do not modify the individual elements of the list. Hereinafter, the principles and embodiments of the disclosure will be described in detail with reference to the accompanying drawings.

In the present specification, an “image” may include a medical image obtained by a magnetic resonance imaging (MRI) apparatus, a computed tomography (CT) apparatus, an ultrasound imaging apparatus, an X-ray apparatus, or another medical imaging apparatus.

Furthermore, in the present specification, an “object” may be a target to be imaged and include a human, an animal, or a part of a human or animal. For example, the object may include a body part, for example an organ, or a phantom.

An MRI system acquires an MR signal and reconstructs the acquired MR signal into an image. The MR signal denotes a radio frequency (RF) signal emitted from the object.

In the MRI system, a main magnet creates a static magnetic field to align a magnetic dipole moment of a specific atomic nucleus of the object placed in the static magnetic field along a direction of the static magnetic field. A gradient coil may generate a gradient magnetic field by applying a gradient signal to a static magnetic field and induce resonance frequencies differently according to each region of the object.

An RF coil may emit an RF signal to match a resonance frequency of a region of the object whose image is to be acquired. Furthermore, when gradient magnetic fields are applied, the RF coil may receive MR signals having different resonance frequencies emitted from a plurality of regions of the object. Though this process, the MRI system may obtain an image from an MR signal by using an image reconstruction technique.

In the present specification, a “user interface” includes an interface that allows a user or operator to interact with a computer or a computer system. The user interface may be configured to provide or receive information or data to or from the user. The user interface may receive an input by the user via a computer and provide an output of the computer to the user.

Furthermore, a “display” or a “display unit” is a device for displaying an image or information related to medical imaging to a user or object. The display may display visual, auditory and/or tactile data. Examples of the display may include a computer monitor, a TV screen, a touch screen, etc.

Throughout the disclosure, the expression “at least one of a, b or c” indicates only a, only b, only c, both a and b, both a and c, both b and c, all of a, b, and c, or variations thereof.

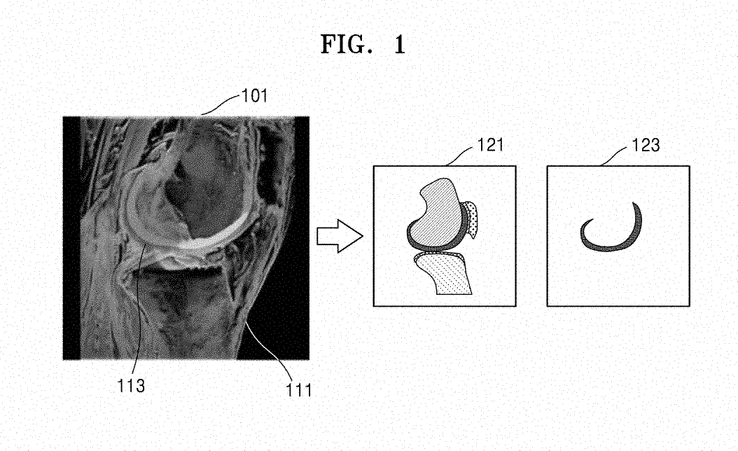

FIG. 1 is a schematic diagram for explaining a medical image generated using a medical imaging application, according to an embodiment.

A medical imaging application may provide various graphical user interfaces (GUIs) designed to improve user convenience for analysis or post-processing of a medical image. For example, the medical imaging application may provide GUI icons that take the shape of a structure of interest (SOI) included in a medical image 101 of an object for selection or post-processing of the SOI.

The medical image 101 may be a rendered three-dimensional (3D) image that is obtained based on medical imaging data acquired using various imaging modalities such as MRI, CT scan, ultrasound imaging, X-ray imaging, etc. A 3D medical image is an image corresponding to a specific cross-section, which is obtained based on volume data acquired using medical imaging, and may be represented in three-dimensions by adding depth information such that perception of depth is provided by applying an image rendering technique.

The medical imaging application may provide icons 121 and 123 that are generated based on at least one SOI recognized in the medical image 101.

For example, when a user receives an input of selecting the medical image 101, the medical imaging application may provide the icons 121 and 123 generated based on at least one SOI recognized in the medical image 101.

For example, when the medical image 101 is an image of a knee, the icon 121 may correspond to SOls 111 of the entire knee recognized in the medical image 101 while the icon 123 may correspond to cartilage 113 recognized therein.

In this way, according to an embodiment, the medical imaging application may provide the icons 121 and 123 having shapes similar to SOls shown in a real image so that the user may intuitively recognize the icons 121 and 123. Furthermore, according to an embodiment, when operations such as enlargement, reduction, translation, rotation, depth value adjustment, brightness adjustment, etc. are performed on the medical image 101 during analysis of the medical image 101, the shapes of the icons 121 and 123 may change as the medical image 101 changes. In this case, it is possible to provide the icons 121 and 123 that may intuitively match shapes of their corresponding real SOls.

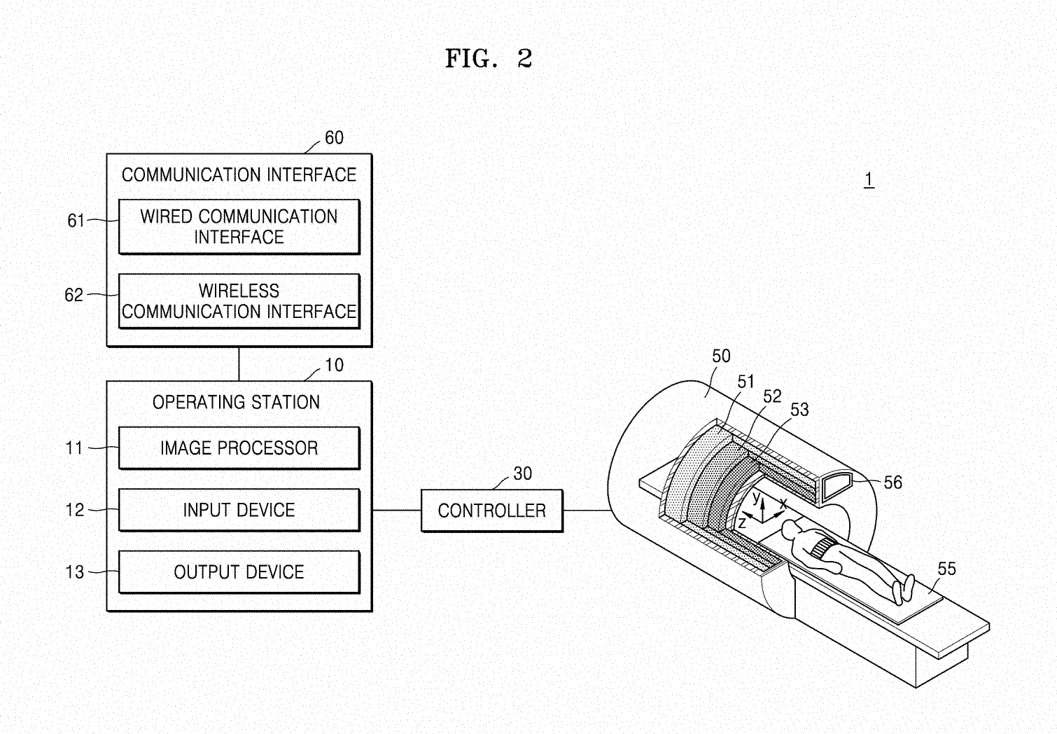

FIG. 2 is a schematic diagram of an MRI system 1.

Referring to FIG. 2, the MRI system 1 may include an operating station 10, a controller 30, and a scanner 50. The controller 30 may be independently separated from the operating station 10 and the scanner 50. Furthermore, the controller 30 may be separated into a plurality of sub-components and incorporated into the operating station 10 and the scanner 50 in the MRI system 1. Operations of the components in the MRI system 1 will now be described in detail.

The scanner 50 may be formed to have a cylindrical shape, for example, a shape of a bore, having an empty inner space into which an object may be inserted. A static magnetic field and a gradient magnetic field are created in the inner space of the scanner 50, and an RF signal is emitted toward the inner space.

The scanner 50 may include a static magnetic field generator 51, a gradient magnetic field generator 52, an RF coil 53, a table 55, and a display 56. The static magnetic field generator 51 creates a static magnetic field for aligning magnetic dipole moments of atomic nuclei of the object in a direction of the static magnetic field. The static magnetic field generator 51 may be formed as a permanent magnet or superconducting magnet using a cooling coil.

The gradient magnetic field generator 52 is connected to the controller 30 and generates a gradient magnetic field by applying a gradient to a static magnetic field in response to a control signal received from the controller 30. The gradient magnetic field generator 52 includes X, Y, and Z coils for generating gradient magnetic fields in X-, Y-, and Z-axis directions crossing each other at right angles and generates a gradient signal according to a position of a region being imaged so as to differently induce resonance frequencies according to regions of the object.

The RF coil 53 may emit an RF signal toward the object in response to a control signal received from the controller 30 and receive an MR signal emitted from the object. In detail, the RF coil 53 may transmit, toward atomic nuclei of the object having precessional motion, an RF signal having the same frequency as that of the precessional motion, stop transmitting the RF signal, and then receive an MR signal emitted from the object.

The RF coil 53 may be formed as a transmitting RF coil for generating an electromagnetic wave having an RF corresponding to the type of an atomic nucleus, a receiving RF coil for receiving an electromagnetic wave emitted from an atomic nucleus, or one transmitting/receiving RF coil serving both functions of the transmitting RF coil and receiving RF coil. Furthermore, in addition to the RF coil unit 53, a separate coil may be attached to the object. Examples of the separate coil may include a head coil, a spine coil, a torso coil, and a knee coil according to a region being imaged or to which the separate coil is attached.

The display 56 may be disposed outside and/or inside the scanner 50. The display 56 is also controlled by the controller 30 to provide a user or the object with information related to medical imaging.

Furthermore, the scanner 50 may include an object monitoring information acquisition unit configured to acquire and transmit monitoring information about a state of the object. For example, the object monitoring information acquisition unit may acquire monitoring information related to the object from a camera for capturing images of a movement or position of the object, a respiration measurer for measuring the respiration of the object, an ECG measurer for measuring the electrical activity of the heart, or a temperature measurer for measuring a temperature of the object and transmit the acquired monitoring information to the controller 30. The controller 30 may in turn control an operation of the scanner 50 based on the monitoring information. Operations of the controller 30 will now be described in more detail.

The controller 30 may control overall operations of the scanner 50.

The controller 30 may control a sequence of signals formed in the scanner 50. The controller 30 may control the gradient magnetic field generator 52 and the RF coil unit 53 according to a pulse sequence received from the operating station 10 or a designed pulse sequence.

A pulse sequence may include all pieces of information required to control the gradient magnetic field generator 52 and the RF coil unit 53. For example, the pulse sequence may include information about a strength, a duration, and application timing of a pulse signal applied to the gradient magnetic field generator 52.

The controller 30 may control a waveform generator for generating a gradient wave, i.e., an electrical pulse according to a pulse sequence and a gradient amplifier for amplifying the generated electrical pulse and transmitting the same to the gradient magnetic field generator 52. Thus, the controller 30 may control formation of a gradient magnetic field by the gradient magnetic field generator 52.

Furthermore, the controller 30 may control an operation of the RF coil unit 53. For example, the controller 30 may supply an RF pulse having a resonance frequency to the RF coil 53 that emits an RF signal toward the object, and receive an MR signal received by the RF coil unit 53. In this case, the controller 30 may adjust emission of an RF signal and reception of an MR signal according to an operating mode by controlling an operation of a switch , for example, a T/R switch, for adjusting transmitting and receiving directions of the RF signal and the MR signal based on a control signal.

The controller 30 may control a movement of the table 55 where the object is placed. Before MRI is performed, the controller 30 may move the table 55 according to which region of the object is to be imaged.

The controller 30 may also control the display 56. For example, the controller 30 control the on/off state of the display 56 or a screen to be output on the display 56 according to a control signal.

The controller 30 may be formed as an algorithm for controlling operations of the components in the MRI system 1, a memory for storing data in the form of a program, and a processor for performing the above-described operations by using the data stored in the memory. In this case, the memory and the processor may be implemented as separate chips. Alternatively, the memory and processor may be incorporated into a single chip.

The operating station 10 may control overall operations of the MRI system 1 and include an image processor 11, an input device 12, and an output device 13.

The image processor 11 may control the memory to store an MR signal received from the controller 30, and generate image data with respect to the object from the stored MR signal by applying an image reconstruction technique.

For example, when a k space, which may also be referred to as a Fourier space or a frequency space, of the memory is filled with digital data to complete k-space data, the image processor 11 may reconstruct image data from the k-space data by applying various image reconstruction techniques, for example, by performing inverse Fourier transform on the k-space data.

Furthermore, the image processor 11 may perform various signal processing operations on MR signals in parallel. For example, the image processor 11 may perform signal processing on a plurality of MR signals received via a multi-channel RF coil in parallel so as to convert the plurality MR signals into image data. In addition, the image processor 11 may store not only the image data in the memory, or the controller 30 may store the same in an external server via a communication interface 60 as will be described below.

The input device 12 may receive, from the user, a control command for controlling the overall operations of the MRI system 1. For example, the input device 12 may receive, from the user, object information, parameter information, a scan condition, and information about a pulse sequence. The input device 12 may be a keyboard, a mouse, a track ball, a voice recognizer, a gesture recognizer, a touch screen, or any other input device.

The output device 13 may output image data generated by the image processor 11. The output device 13 may also output a user interface (UI) configured so that the user may receive a control command related to the MRI system 1. The output device 13 may be formed as a speaker, a printer, a display, or any other output device.

Furthermore, although FIG. 2 shows that the operating station 10 and the controller 30 are separate components, the operating station 10 and the controller 30 may be included in a single device as described above. Furthermore, processes respectively performed by the operating station 10 and the controller 30 may be performed by another component. For example, the image processor 11 may convert an MR signal received from the controller 30 into a digital signal, or the controller 30 may directly perform the conversion of the MR signal into the digital signal.

The MRI system 1 may further include a communication interface 60 and be connected to an external device such as a server, a medical apparatus, and a portable device, for example, a smartphone, a tablet PC, a wearable device, etc., via the communication interface 60.

The communication interface 60 may include at least one component that enables communication with an external device. For example, the communication interface 60 may include at least one of a local area communication module, a wired communication interface 61, or a wireless communication interface 62.

The communication interface 60 may receive a control signal and data from an external device and transmit the received control signal to the controller 30 so that the controller 30 may control the MRI system 1 according to the received signal.

Alternatively, by transmitting a control signal to an external device via the communication interface 60, the controller 30 may control the external device according to the control signal.

For example, the external device may process data according to a control signal received from the controller 30 via the communication interface 60.

A program for controlling the MRI system 1 may be installed on the external device and may include instructions for performing some or all of the operations of the controller 30.

The program may be preinstalled on the external device, or a user of the external device may download the program from a server providing an application for installation. The server providing an application may include a recording medium having the program recorded thereon.

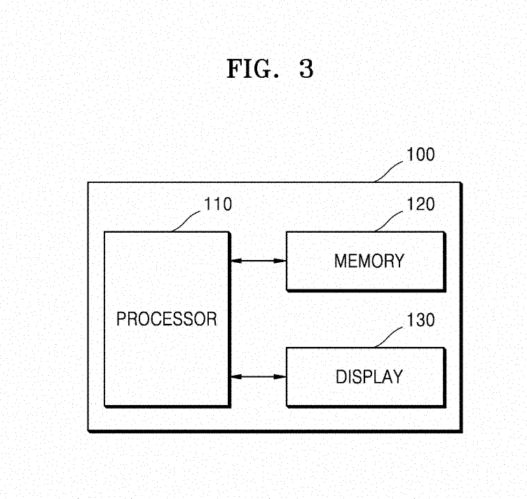

FIG. 3 is a block diagram of a configuration of a medical imaging apparatus 100 according to an embodiment.

According to an embodiment, the medical imaging apparatus 100 may include any image processing apparatus capable of obtaining a medical image based on medical image data acquired using medical imaging. In detail, the medical imaging apparatus 100 may include a computing device for displaying an image in real-time based on data acquired using medical imaging or an image generated by post-processing data acquired using medical imaging.

Furthermore, the medical imaging apparatus 100 may be an apparatus having functions of receiving medical image data acquired using medical imaging, processing the medical image data, and displaying a resulting image. For example, the medical imaging apparatus 100 may be an apparatus capable of displaying an image obtained by processing volume data acquired by an MRI apparatus, but is not limited thereto. The medical imaging apparatus 100 may include an apparatus capable of displaying a medical image such as a CT or ultrasound image.

Furthermore, the medical imaging apparatus 100 may include a medical server apparatus in a hospital where a patient undergoes medical imaging or in another hospital, but is not limited thereto. Examples of the medical imaging apparatus 100 may include a smartphone, a tablet personal computer (PC), a PC, a smart television (TV), a mobile phone, a personal digital assistant (PDA), a laptop, a media player, a digital camera, a home appliance, and other mobile or non-mobile computing devices.

Referring to FIG. 3, the medical imaging apparatus 100 according to the embodiment may include a processor 110, a memory 120, and a display 130.

The medical imaging apparatus 100 may be included in the MRI system 1 described with reference to FIG. 2. In this case, the display 130 of the medical imaging apparatus 100 may correspond to the output device 13 shown in FIG. 2. The processor 110 and the memory 120 may correspond to one or a combination of the image processor 11 and the controller 30 described with reference to FIG. 2.

According to an embodiment, the processor 110 may execute one or more instructions or applications stored in the memory 120. In this case, an application may be an analysis application providing tools for analyzing a medical image.

The processor 110 may generate a rendered 3D medical image of a region of interest (ROI) based on medical imaging data. Medical imaging data may include pieces of data acquired by various types of medical imaging apparatuses such as MRI equipment, a CT apparatus, an ultrasound device, an X-ray apparatus, etc.

For example, the processor 110 may be configured to obtain an MR image based on MR signal data stored in the memory 120 or received from an external device. For example, the MR signal data may include MR signals received from a scanner. In this case, the MR signal data may be MR image data with respect to a plurality of slices acquired using an MRI scan.

Furthermore, medical imaging data may include volume data acquired using a CT scan, ultrasound imaging, and X-ray imaging.

The processor 110 may generate a rendered 3D medical image of an ROI based on medical imaging data and information related to medical imaging. The information related to medical imaging may include pieces of information about an object and pieces of information about parts of the ROI, and may be contained in a header of a medical image file corresponding to the medical imaging data.

According to an embodiment, a medical image file may include a header and medical imaging data. The header may include information about the medical image file, and the medical imaging data may include medical image data with respect to the object. The information about the medical image file contained in the header may be object type information, medical image data acquisition modality information, or the like. The object type information may include information about an ROI of the object, e.g., an anatomical structure of the object such as the heart, knee joint, spine, brain, etc., but is not particularly limited thereto. A medical image data acquisition modality may be X-ray, CT, MRI, ultrasound, or the like, but is not particularly limited thereto. The header may further include an imaging date, personal information about a patient, etc. The medical image file may be a file compliant with a Digital Imaging and Communications in Medicine (DICOM) standard.

The processor 110 may recognize or identify at least one SOI included in a 3D medical image and generate at least one SOI icon including a first image showing the at least one SOI.

In an embodiment, a first image is an image obtained by reducing at least a portion of the 3D medical image and may have the same view as a selected view. Furthermore, the first image may include a figure representing its corresponding SOI. The at least one SOI may correspond to an anatomical region depicted in the 3D medical image. For example, when the ROI or the 3D medical image includes an anatomical structure, the processor 110 may identify the anatomical structure, and may recognize or identify one or more anatomical sub-structures included in the anatomical structure. The processor may determine the anatomical sub-structures to be the one or more SOI.

The memory 120 may store various pieces of data, programs, or applications for driving and controlling the medical imaging apparatus 100. A program stored in the memory 120 may include one or more instructions. The programs or applications stored in the memory 120 may be executed by the processor 110. The program stored in the memory 120 may be downloaded or downloadable to another device. Furthermore, the program may be downloadable by a remote data processing system such that the program may be used on the remote data processing system as well as on the memory 120.

The display 130 may output a UI configured such that the user may input a control command related to the medical imaging apparatus 100. Furthermore, the display 130 may display at least one SOI icon according to control by the processor 110.

The display 130 may display a resulting image rendered based on medical imaging data, together with at least one SOI icon. Furthermore, the display 130 may output information needed for the user to manipulate the medical imaging apparatus 100, such as user information or object information. Examples of the display 130 may include a cathode-ray tube (CRT) display, a liquid crystal display (LCD), a plasma display panel (PDP), an organic light-emitting display (OLED), a field emission display (FED), a light-emitting diode (LED) display, a vacuum fluorescent display (VFD), a digital light processing (DLP) display, a flat panel display (FPD), a 3D display, a transparent display, and various other displays as desired.

The medical imaging apparatus 100 may further include an input device. The medical imaging apparatus 100 may receive a control command from the user via the input device and perform an image processing function corresponding to a selected icon. Examples of the input device may include a keyboard, a mouse, a track ball, a voice recognizer, a gesture recognizer, a touch screen, a stylus pen, and various other input devices as desired. For example, in an embodiment in which the display 130 is formed as a touch screen, the display 130 may function as the input device.

Components of the medical imaging apparatus 100 are not limited to those shown in FIG. 3. According to an embodiment, the medical imaging apparatus 100 may include more or fewer components than those shown in FIG. 3, and may not include some of the components shown therein.

For example, the medical imaging apparatus 100 may further include a communication unit including at least one component that enables communication with at least one of a client device, an external server, or an external database.

The communication unit may be connected to a network by wire or wirelessly and communicates with an external device or server. The communication unit may exchange data with a server or another medical device in a hospital, which is connected via a picture archiving and communication system (PACS). Furthermore, the communication unit may perform data communication according to the DICOM standard.

Furthermore, the medical imaging apparatus 100 may not include the display 130 but be connected to a separate external display to be operated.

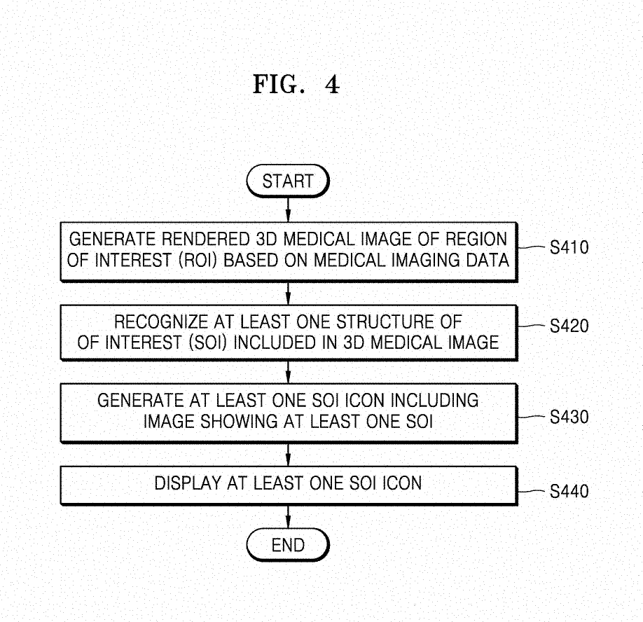

FIG. 4 is a flowchart of a method of controlling a medical imaging apparatus, which may correspond to medical imaging apparatus 100 of FIG. 3, according to an embodiment.

The medical imaging apparatus 100 may generate a rendered 3D medical image of an ROI based on medical imaging data at operation S410.

For example, the medical imaging apparatus 100 may generate a rendered 3D medical image based on MR image data with respect to a plurality of slices acquired during an MRI scan. Furthermore, the medical imaging apparatus 100 may generate a rendered 3D medical image based on volume data acquired using a CT scan, ultrasound imaging, and X-ray imaging.

The rendered 3D medical image may be obtained using a photorealistic rendering technique whereby an ROI is represented as being similar to a real photograph based on acquired medical image data with respect to the ROI. Furthermore, the rendered 3D medical image may be obtained using any desired rendering techniques, and may include texture mapping, lighting, and shading information therein.

Furthermore, the rendered 3D medical image may be obtained based on information about medical imaging as well as the medical imaging data. According to an embodiment, the rendered 3D medical image may be obtained based on medical imaging data and information about a part of the ROI.

The medical imaging apparatus 100 may recognize at least one SOI included in the rendered 3D medical image at operation S420.

For example, the medical imaging apparatus 100 may segment the 3D medical image into a plurality of regions. The medical imaging apparatus 100 may then recognize an SOI based on a result of segmenting the rendered 3D medical image. The medical imaging apparatus 100 may segment the 3D medical image into at least one SOI based on the rendered 3D medical image.

Furthermore, the medical imaging apparatus 100 may segment the 3D medical image into at least one SOI based on the medical imaging data. For example, the medical imaging apparatus 100 may segment the 3D medical image into at least one SOI, based on data acquired by segmenting an image corresponding to each of a plurality of slices acquired during an MRI scan into at least one SOI.

Furthermore, the medical imaging apparatus 100 may segment the 3D medical image into at least one SOI by using any desired image segmentation techniques.

In addition, the medical imaging apparatus 100 may segment the rendered 3D medical image into at least one SOI based on information about medical imaging. According to an embodiment, the rendered 3D medical image may be partitioned into at least one SOI based on medical imaging data and information about a part of the ROI.

For example, when the rendered 3D medical image includes a knee, the medical imaging apparatus 100 may segment the rendered 3D medical image into femoral cartilage, tibial cartilage, meniscus, patellar cartilage, and kneecap. For example, the medical imaging apparatus 100 may identify the knee as an anatomical structure, and may identify the femoral cartilage, tibial cartilage, meniscus, patellar cartilage, and kneecap as anatomical sub-structures included in the knee. The medical imaging apparatus 100 may determine these anatomical sub-structures to be SOls.

As another example, when the rendered 3D medical image shows a left ventricle of the heart, the medical imaging apparatus 100 may segment the rendered 3D medical image into inner and outer myocardial walls of the left ventricle.

The medical imaging apparatus 100 may generate at least one SOI icon, each including an image showing the at least one SOI at operation S430.

For example, the medical imaging apparatus 100 may generate at least one SOI icon, each including an image showing at least one SOI, in response to an input for generating the rendered 3D medical image.

An image showing at least one SOI may be obtained by reducing at least a portion of the 3D medical image.

Furthermore, an image showing the at least one SOI may be obtained by reducing a portion corresponding to the at least one SOI in the 3D medical image, and may have the same view as that of the 3D medical image.

Furthermore, an image showing at least one SOI may include a figure that takes the shape of the SOI. The at least one SOI may correspond to an anatomical region depicted in the 3D medical image.

The medical imaging apparatus 100 may display the at least one SOI icon at S440.

The medical imaging apparatus 100 may display the at least one SOI icon together with other icons having image processing functions.

A process whereby the medical imaging apparatus 100 generates and displays at least one SOI icon, each including an image showing at least one SOI, according to an embodiment will now be described.

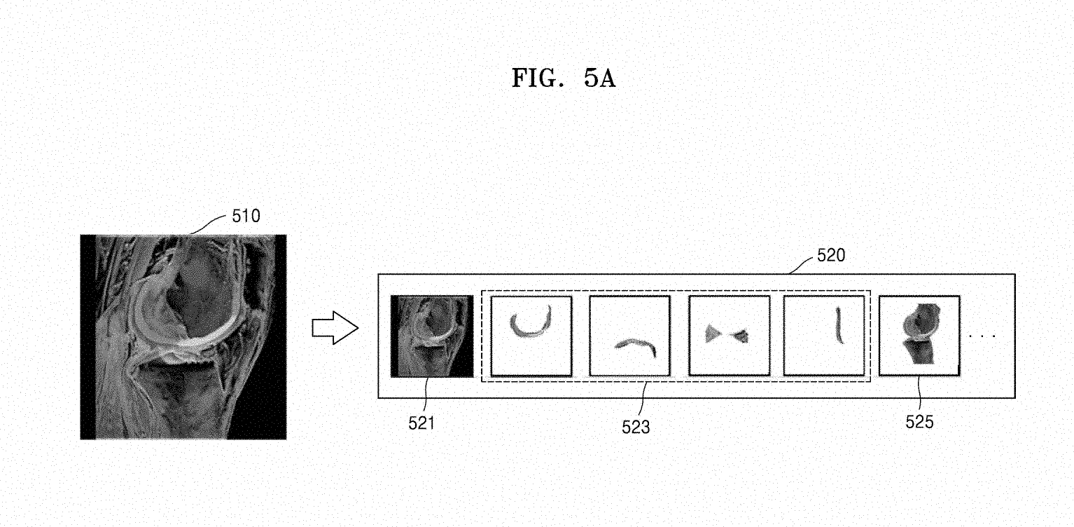

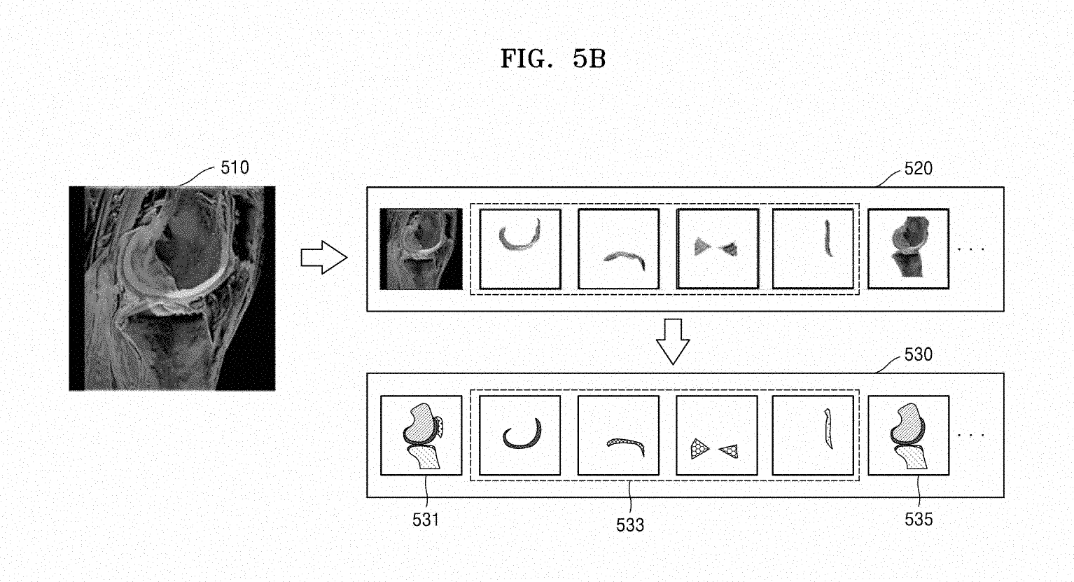

FIG. 5A illustrates icons generated based on at least one SOI.

Referring to FIG. 5A, the medical imaging apparatus 100 may provide an SOI icon corresponding to a shape of an SOI for selecting or post-processing the SOI included in a medical image 510. The medical image 510 may be a 3D rendered image. At least one SOI may be an anatomical region depicted in a 3D medical image.

According to an embodiment, when an input of selecting the medical image 510 is received, the medical imaging apparatus 100 may generate at least one first SOI icon 520 based on at least one SOI recognized in the medical image 510.

Furthermore, the medical imaging apparatus 100 may generate the at least one first SOI icon 520 based on at least one SOI recognized in the medical image 510 and information about medical imaging.

The information about medical imaging may include information about an ROI of an object and information about which view is used to acquire a medical image. For example, an ROI of an object may include a knee joint, the heart, the brain, etc. Views for the object may include a sagittal plane view, a coronal plane view, an axial plane view, and other views corresponding to cross-sections desired to be observed. The information about medical imaging may be contained in a header of a medical image file corresponding to medical imaging data.

For example, the information about medical imaging may include information indicating that an ROI of the object in the medical image 510 is a knee and the medical image 510 is acquired in a sagittal plane view. The medical imaging apparatus 100 may generate the at least one first SOI icon 520 based on information about at least one SOI recognized in the medical image 510 and information indicating that the medical image 510 is an image of a knee in a sagittal plane view.

According to an embodiment, the at least one first SOI icon 520 may include at least one first image.

Each first image may correspond to at least one SOI in a cross-section of interest of the medical image 510 and have the same view as a selected view. In detail, the first image may show at least one SOI in a cross-section of interest and have a different resolution than an image corresponding to the cross-section of interest of the medical image 510. The first image may also have a lower resolution and a smaller size than the medical image 510.

For example, when the medical image 510 includes a knee joint, a first image included in an SOI icon 521 may show the entire knee joint included in the medical image 510.

Furthermore, when the medical image 510 includes the knee joint, first images in SOI icons 523 may each show only one of femoral cartilage, tibial cartilage, meniscus, and patellar cartilage included in the medical image 510.

In addition, when the medical image 510 includes the knee joint, a first image in an SOI icon 525 may show the bone included in the medical image 510.

FIG. 5B illustrate icons generated based on at least one SOI. Referring to FIG. 5B, the medical imaging apparatus 100 may provide at least one second SOI icon 530 corresponding to SOls in the medical image 510. A first image in the at least one second SOI icon 530 may include a figure representing at least one SOI. The at least one first image in the at least one second SOI icon 530 may be a figure showing SOls in different colors.

The at least one second SOI icon 530 may be generated based on the at least one first SOI icon 520.

For example, an SOI icon 531 included in the at least one second SOI icon 530 may be generated based on the SOI icon 521 and include a figure representing the entire knee joint in the medical image 510.

Furthermore, SOI icons 533 included in the at least one second SOI icon 530 may be generated based on the SOI icons 523 and may each include a figure representing only one of femoral cartilage, tibial cartilage, meniscus, and patellar cartilage included in the medical image 510.

In addition, an SOI icon 535 included in the at least one second SOI icon 530 may be generated based on the SOI icon 525 and include a figure representing the bone in the medical image 510.

Because a shape of an SOI in a rendered 3D medical image may vary according to views or planes of the rendered 3D medical image, the medical imaging apparatus 100 may provide SOI icons including first images showing different shapes so that each SOI icon may intuitively match its corresponding SOI included in the 3D medical image.

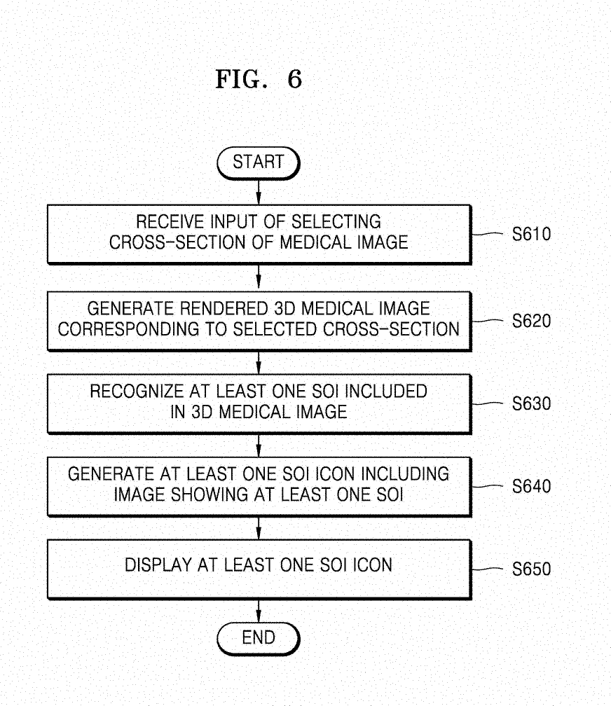

FIG. 6 is a flowchart of a method of controlling the medical imaging apparatus 100, according to an embodiment.

The medical imaging apparatus 100 may receive an input of selecting a cross-section of a medical image at operation S610.

An input of selecting a cross-section of a medical image may include an input of selecting at least one from among a plurality of slices corresponding to acquired medical imaging data. Furthermore, the input of selecting a cross-section of a medical image may include an input of selecting a view of a 3D medical image. In addition, the input of selecting a medical image may include an input of selecting at least one cross-section desired to be observed from an image displayed by the medical imaging apparatus 100.

The medical imaging apparatus 100 may generate a rendered 3D medical image corresponding to the selected cross-section at operation S620.

The medical imaging apparatus 100 may generate, based on acquired imaging data, a rendered 3D medical image corresponding to a selected cross-section.

Furthermore, the medical imaging apparatus 100 may generate a rendered 3D medical image corresponding to a selected cross-section, based on information about medical imaging and medical imaging data.

The information about medical imaging may include at least one of information about an ROI of an object included in the selected cross-section, information about which of a plurality of slices corresponds to the selected cross-section, or information about which view corresponds to the selected cross-section.

The medical imaging apparatus 100 may generate a rendered 3D medical image corresponding to a selected cross-section, based on medical imaging data and information about medical imaging, which includes at least one of information about an ROI of an object included in the selected cross-section, information about which of a plurality of slices corresponds to the selected cross-section, or information about which view corresponds to the selected cross-section.

The medical imaging apparatus 100 may recognize at least one SOI included in the rendered 3D medical image at operation S630.

The medical imaging apparatus 100 may recognize at least one SOI included in the 3D medical image based on information about medical imaging.

For example, the medical imaging apparatus 100 may recognize, based on information indicating that the rendered 3D medical image includes a knee, SOls included in the 3D medical image as being femoral cartilage, tibial cartilage, meniscus, kneecap, etc.

As another example, the medical imaging apparatus 100 may recognize SOls included in the 3D medical image as being inner and outer myocardial walls, based on information indicating that the rendered 3D medical image shows the left ventricle of the heart.

The medical imaging apparatus 100 may generate at least one SOI icon, each including an image showing the at least one SOI at operation S640.

The medical imaging apparatus 100 may generate at least one SOI icon, each including an image showing at least one SOI, in response to an input for generating the rendered 3D medical image corresponding to the selected cross-section.

For example, the medical imaging apparatus 100 may render, based on a specific event including the user clicking a mouse to select a cross-section, a 3D medical image corresponding to the selected cross-section, recognize at least one SOI included in the 3D medical image, and generate at least one SOI icon based on the recognized at least one SOI.

The medical imaging apparatus 100 may display the at least one SOI icon at operation S650.

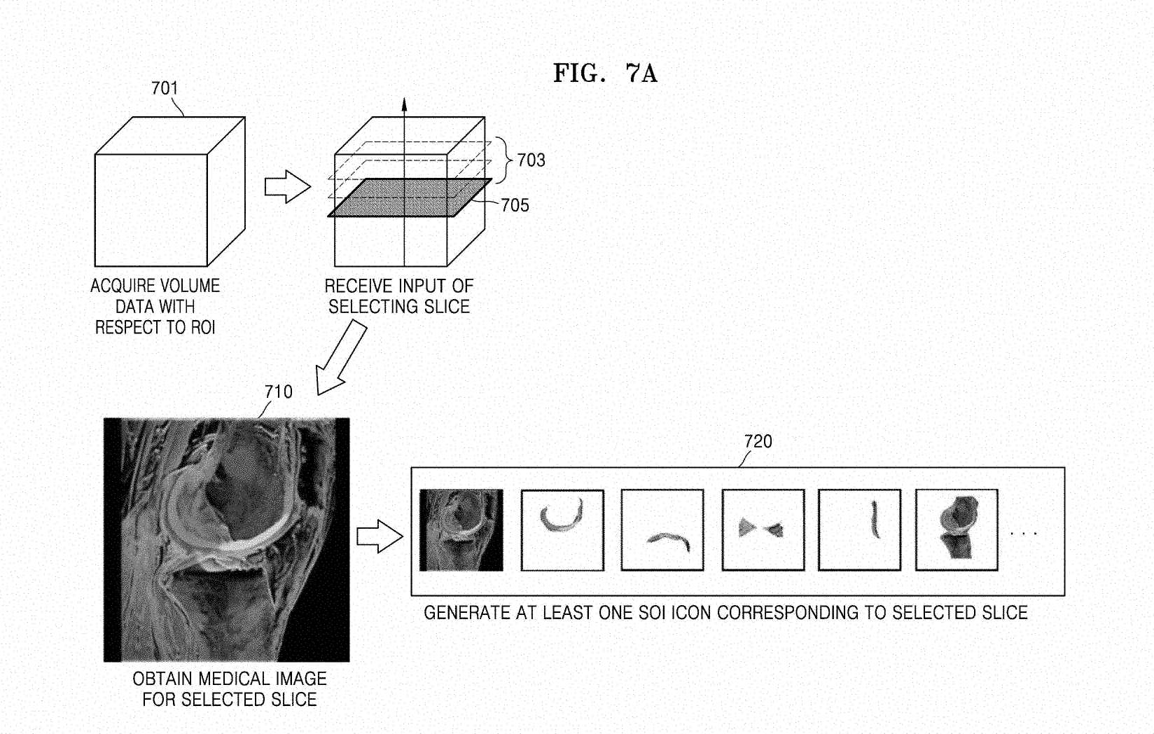

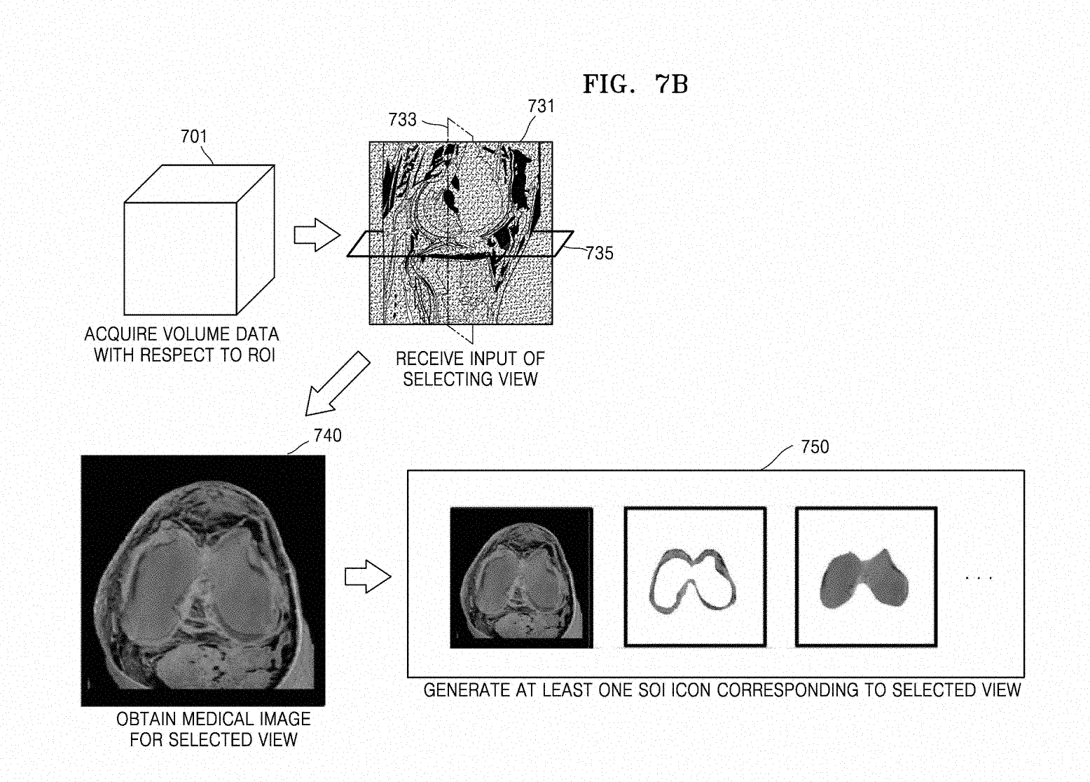

FIG. 7A illustrates generation of icons corresponding to a cross-section in a medical image based on an input of selecting the cross-section.

Referring to FIG. 7A, the medical imaging apparatus 100 may acquire medical imaging data 701 with respect to an ROI.

The medical imaging data 701 may include pieces of data acquired by various types of medical imaging apparatuses such as MRI equipment, a CT apparatus, an ultrasound device, an X-ray apparatus, etc. Furthermore, the medical imaging data 701 may include 3D volume data with respect to the ROI or cross-sectional image data with respect to a plurality of slices.

An input of selecting a cross-section of a medical image may include an input of selecting at least one slice 705 from among a plurality of slices 703 corresponding to the acquired medical imaging data 701.

When an input of selecting a slice for a 3D medical image is received, the medical imaging apparatus 100 may obtain a rendered 3D medical image 710 of the ROI based on the medical imaging data 701 and the selected slice 705.

The medical imaging apparatus 100 may recognize at least one SOI included in the obtained 3D medical image. For example, the medical imaging apparatus 100 may recognize at least one SOI included in the 3D medical image based on information contained in the medical imaging data 701. The medical imaging data 701 may include information about a result of segmenting an image corresponding to each of the plurality of slices into at least one SOI.

The medical imaging apparatus 100 may generate at least one SOI icon 720, each including a first image showing at least one SOI included in the 3D medical image 710 corresponding to the selected slice.

Furthermore, the input of selecting a cross-section of a medical image may include an input for changing a medical image corresponding to a slice currently being displayed by the medical imaging apparatus 100 to a medical image corresponding to another slice. When an input for changing a current slice for a 3D medical image to another slice is received, the medical imaging apparatus 100 may update the rendered 3D medical image 710 of the ROI based on the medical imaging data 701 and the other slice. Furthermore, the medical imaging apparatus 100 may update the at least one SOI icon 720 with respect to the other slice.

FIG. 7B illustrates generation of icons corresponding to a cross-section in a medical image based on an input of selecting the cross-section. Referring to FIG. 7B, an input of selecting a cross-section of a medical image may include an input for selecting a view of a 3D medical image 731, which is desired to be observed, from acquired medical imaging data 701).

The medical imaging apparatus 100 may recognize at least one SOI included in an obtained 3D medical image. For example, the medical imaging apparatus 100 may recognize at least one SOI included in a 3D medical image based on information contained in the medical imaging data 701. The medical imaging data 701 may include information about a result of segmenting an image corresponding to each of a plurality of slices into at least one SOI.

The medical imaging apparatus 100 may generate at least one SOI icon 750, each including a first image showing at least one SOI included in a rendered 3D medical image 740 corresponding to a selected view.

Furthermore, the input of selecting a cross-section of a medical image may include an input of changing a first view 733 of a cross-section of a 3D medical image 731 currently being displayed by the medical imaging apparatus 100 to a second view 735. An input of changing a desired view of the 3D medical image 731 may be an input of changing a position of a mark indicating the first view 733 to a position indicating the second view 735 in the 3D medical image 731 including an ROI.

When an input of changing a view of the 3D medical image 731 is received, the medical imaging apparatus 100 may obtain the rendered 3D medical image 740 of the ROI based on the medical imaging data 701 and a changed result, i.e., the second view 735. Furthermore, the medical imaging apparatus 100 may update the at least one SOI icon 750, each including a first image showing at least one SOI, based on the medical imaging data 701 and the second view 735.

According to an embodiment, the input of selecting a cross-section of a medical image may further include an input for zooming in or out the rendered 3D medical image 740 corresponding to the selected view.

When an input for zooming in or out the rendered 3D medical image 740 is received, the medical imaging apparatus 100 may update the rendered 3D medical image 740 of the ROI based on the medical imaging data 701 and the input. The medical imaging apparatus 100 may also update the at least one SOI icon 750 included in the updated rendered 3D medical image 740, based on the input for zooming in or out the rendered 3D medical image 740.

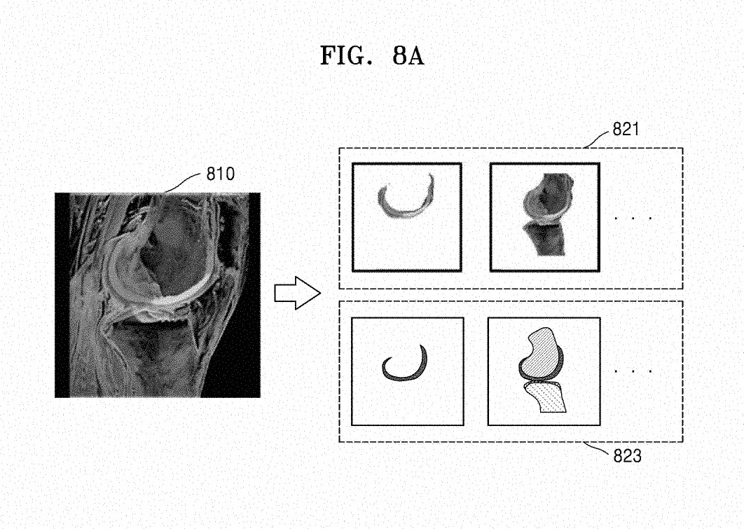

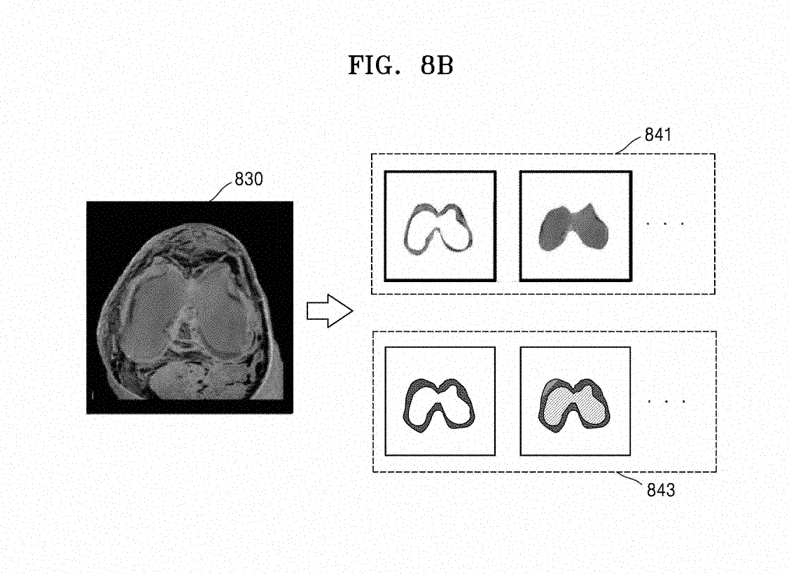

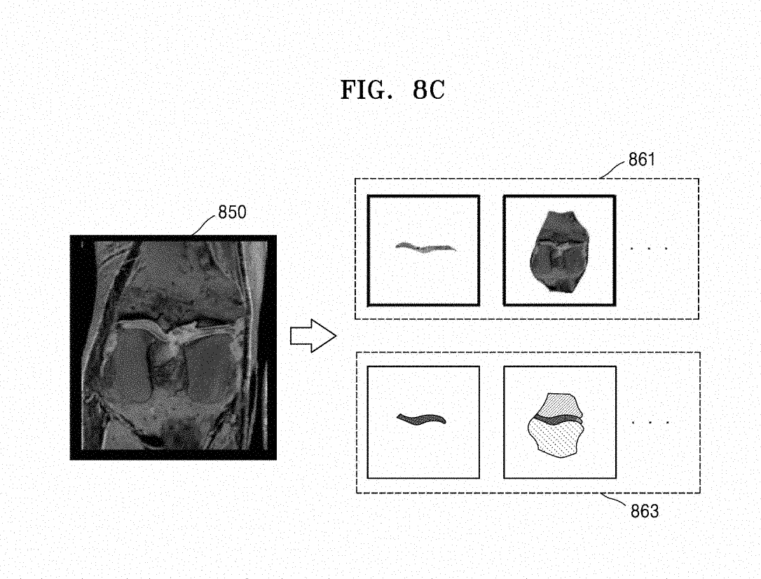

FIG. 8A illustrates at least one SOI icon that changes according to a view of a medical image, according to an embodiment.

Referring to FIG. 8A, when a sagittal plane view is selected as a view of a 3D medical image, at least one first SOI icon 821 and at least one second SOI icon 823 may be generated based on a rendered 3D medical image 810 in the sagittal plane view.

For example, when an ROI includes a knee, the at least one first SOI icon 821 may include an icon representing only a specific cartilage, an icon representing the entire bone, etc. The at least one second SOI icon 823 may include a figure generated based on a first image included in the at least one first SOI icon 821.

FIG. 8B illustrates at least one SOI icon that changes according to a view of a medical image, according to an embodiment. Referring to FIG. 8B, when a coronal plane view is selected as a view of a 3D medical image, at least one first SOI icon 841 and at least one second SOI icon 843 may be generated based on a rendered 3D medical image 830 in the coronal plane view.

FIG. 8C illustrates at least one SOI icon that changes according to a view of a medical image, according to an embodiment. Referring to FIG. 8C, when an axial plane view is selected as a view of a 3D medical image, at least one first SOI icon 861 and at least one second SOI icon 863 may be generated based on a rendered 3D medical image 850 in the axial plane view.

Referring to FIGS. 8A through 8C, because a shape of an SOI in a 3D medical image may vary according to desired views of the 3D medical image, the medical imaging apparatus 100 may provide SOI icons having different shapes so that each SOI icon may intuitively match its corresponding SOI included in the 3D medical image.

Furthermore, the medical imaging apparatus 100 may provide a first SOI icon representing a real shape of an SOI included in a rendered 3D medical image based on the rendered 3D medical image, thereby allowing a user to easily recognize an icon included in the first SOI icon.

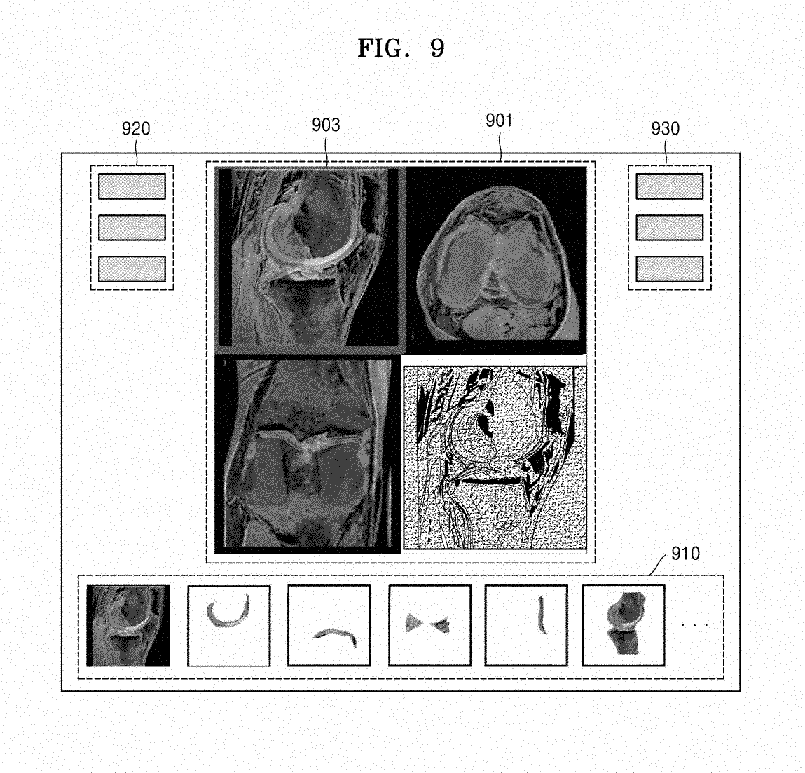

FIG. 9 illustrates a screen displayed by the medical imaging apparatus 100, according to an embodiment.

Referring to FIG. 9, the medical imaging apparatus 100 may generate a plurality of rendered 3D medical images 901 respectively corresponding to a plurality of views. Furthermore, the medical imaging apparatus 100 may generate an icon corresponding to an image selected from among the rendered 3D medical images 901. For example, as shown in FIG. 9, when an image 903 selected by the user from among the rendered 3D medical images 901 is a sagittal plane view image, the medical imaging apparatus 100 may generate and display at least one SOI icon 910 corresponding to the sagittal plane view image.

In addition, the medical imaging apparatus 100 may generate at least one SOI icon 910 for each of the rendered 3D medical images 901. In detail, the medical imaging apparatus 100 may generate in advance SOI icons respectively corresponding to a plurality of views of an ROI, i.e., a sagittal plane view, a coronal plane view, and an axial plane view, and when the user selects the image 903 from among the rendered 3D medical images 901, display the at least one SOI icon 910 corresponding to the image 903.

The medical imaging apparatus 100 may provide sub-icons 920 and 930 having image processing functions for each of the rendered 3D medical images 901, in addition to icons representing SOls. Image processing functions may differ according to ROls of an object respectively included in the rendered 3D medical images 901.

When an ROI of the object includes a knee joint, the at least one SOI icon 910 may include SOI icons respectively representing femoral cartilage, tibial cartilage, meniscus, patellar cartilage, kneecap, etc. In this case, the medical imaging apparatus 100 may further provide the sub-icons 920 and 930 for performing image processing functions such as cartilage segmentation, measurement of cartilage volume or thickness, etc.

When an ROI of the object may include blood vessels, the at least one SOI icon 910 may include SOI icons respectively representing an artery and a vein. In this case, the medical imaging apparatus 100 may further provide the sub-icons 920 and 930 for performing image processing functions such as performing maximal intensity projection (MIP) and classification into an artery and a vein in an image.

When an ROI of the object may include the brain and a patient is diagnosed with cerebral infarction, the at least one SOI icon 910 may include SOI icons respectively representing infarct core and penumbra surrounding he infarct core. In this case, the medical imaging apparatus 100 may further provide the sub-icons 920 and 930 for performing image processing functions such as segmentation and selection of infarct core and penumbra, generation of a mismatch map that is used to compare a diffusion-weighted image (DWI) with a perfusion-weighted image (PWI), ROI statistics, etc.

When an ROI of the object includes the brain and a patient is diagnosed with dementia, the at least one SOI icon 910 may include SOI icons respectively representing white matter, hippocampus, a ventricle, etc. In this case, the medical imaging apparatus 100 may further provide the sub-icons 920 and 930 for performing image processing functions such as segmentation and tracing of white matter hyperintensity, hippocampus, and ventricle, measurement of white matter hyperintensity volume, hippocampal volume, and ventricular volume, etc.

As described above, the medical imaging apparatus 100 may provide the at least one SOI icon 910 corresponding to each of the rendered 3D medical images 901 and the sub-icons 920 and 930 having image processing functions for each of the rendered 3D medical images 901.

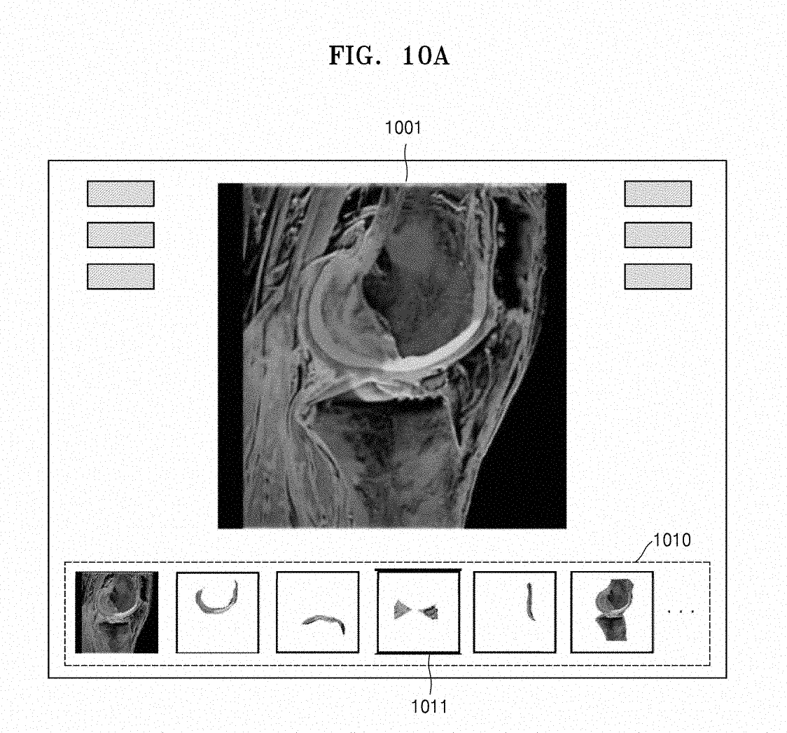

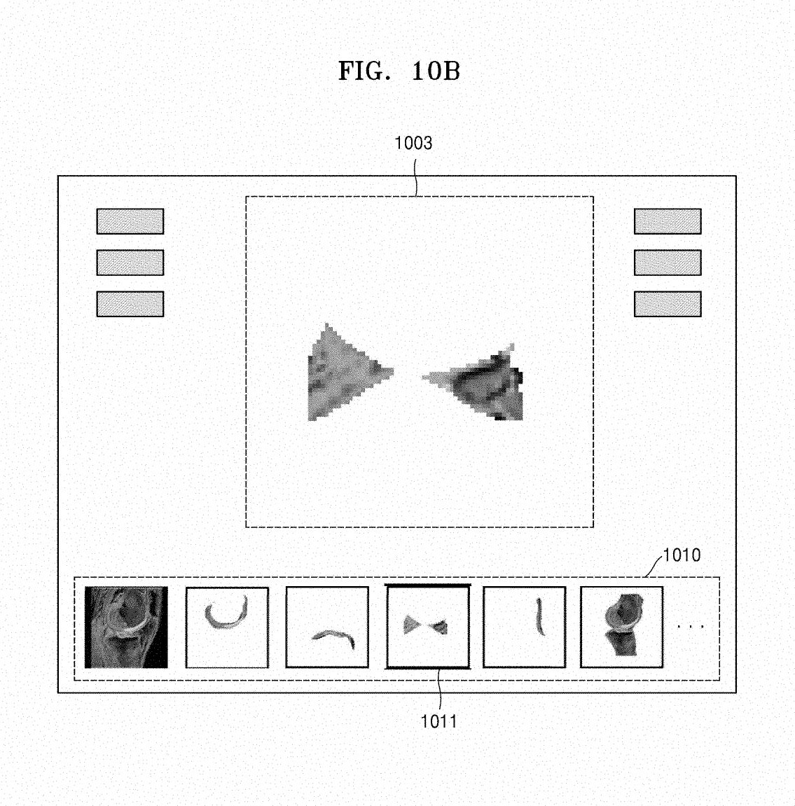

FIG. 10A illustrates a screen displayed by the medical imaging apparatus 100, according to an embodiment.

The medical imaging apparatus 100 may generate a rendered 3D medical image 1001 of an ROI based on medical imaging data. The rendered 3D medical image 1001 may be an image corresponding to a view selected from among a plurality of views.

The medical imaging apparatus 100 may display the rendered 3D medical image 1001 together with at least one SOI icon 1010 corresponding thereto.

The medical imaging apparatus 100 may display an SOI icon 1011 that represents an SOI including a lesion from among the at least one SOI icon 1010 by adding an indication that the lesion is present in the SOI corresponding to the SOI icon 1011.

In detail, the medical imaging apparatus 100 may display the SOI icon 1011 corresponding to an SOI including a lesion by adding, to the SOI icon 1011, an indication that distinguishes the SOI icon 1011 from the other SOI icons. For example, the medical imaging apparatus 100 may display a contour of the SOI icon 1011 corresponding to an SOI including a lesion with bold lines. The medical imaging apparatus 100 may also display a contour of an SOI including a lesion within the rendered 3D medical image 1001.

FIG. 10B illustrates a screen displayed by the medical imaging apparatus 100, according to an embodiment. Referring to FIG. 10B, the medical imaging apparatus 100 may display, based on an input of selecting an SOI icon 1011 corresponding to an SOI including a lesion, an image 1003 showing the SOI including the lesion corresponding to the selected SOI icon 1011. The image 1003 showing the SOI including the lesion may have a larger size and a higher resolution than a first image showing an SOI included in the SOI icon 1011

The medical imaging apparatus 100 may display an SOI including a lesion in the rendered 3D medical image 1001 and the at least one SOI icon 1010 in various ways such that the user may more easily identify a location of the lesion. Furthermore, the medical imaging apparatus 100 may display, based on a user input, an SOI including a lesion according to various views.

Embodiments may be implemented through non-transitory computer-readable recording media having recorded thereon computer-executable instructions and data. The instructions may be stored in the form of program codes, and when executed by a processor, may cause the processor to generate a predetermined program module to perform a specific operation. Furthermore, when being executed by the processor, the instructions may cause the processor to perform specific operations according to the embodiments.

While embodiments have been described with reference to the figures, it will be understood by those of ordinary skill in the art that various changes in form and details may be made therein without departing from the spirit and scope of the disclosure as defined by the following claims. Accordingly, the above embodiments of the disclosure and all aspects thereof are examples only and are not limiting.

What is claimed is:

1. A medical imaging apparatus comprising:
a display;
a memory configured to store one or more instructions; and
a processor configured to execute the one or more instructions to:
acquire a rendered three-dimensional medical image of a region of interest (ROI) based on medical imaging data;
recognize at least one structure of interest (SOI) included in the rendered three-dimensional medical image;
acquire at least one SOI icon including at least one image showing the at least one SOI; and
control the display to display the at least one SOI icon.

2. The medical imaging apparatus of claim 1, wherein the at least one image includes an image obtained by reducing a portion corresponding to the at least one SOI in the rendered three-dimensional medical image or an image including a figure representing the at least one SOI that is acquired based on a result of segmenting the at least one SOI.

3. The medical imaging apparatus of claim 1, wherein the processor is further configured to execute the one or more instructions to acquire the at least one SOI icon in response to an input for acquiring the rendered three-dimensional medical image.

4. The medical imaging apparatus of claim 1, wherein the processor is further configured to execute the one or more instructions to:
based on receiving an input for changing a first slice of the rendered three-dimensional medical image to a second slice, update the rendered three-dimensional medical image of the ROI being based on the medical imaging data and the second slice;
recognize at least one new SOI included in a result of the updating; and
update the at least one image to show the at least one new SOI.

5. The medical imaging apparatus of claim 1, wherein the processor is further configured to execute the one or more instructions to:
based on receiving an input for changing a first view of the rendered three-dimensional medical image to a second view, update the rendered three-dimensional medical image of the ROI based on the medical imaging data and the second view;
recognize at least one new SOI included in a result of the updating; and
update the at least one image to show the at least one new SOI.

6. The medical imaging apparatus of claim 1, wherein the processor is further configured to execute the one or more instructions to:
based on receiving an input for zooming the rendered three-dimensional medical image, update the rendered three-dimensional medical image of the ROI based on the medical imaging data and the input;
recognize at least one new SOI included in a result of the updating; and
update the at least one image to show the at least one new SOI.

7. The medical imaging apparatus of claim 1, wherein the processor is further configured to execute the one or more instructions to:
acquire a plurality of rendered three-dimensional medical images respectively corresponding to a plurality of views; and
acquire the at least one SOI icon corresponding to an image selected by a user from among the plurality of rendered three-dimensional medical images.

8. The medical imaging apparatus of claim 1, wherein the at least one image is acquired based on the medical imaging data and information about medical imaging.

9. The medical imaging apparatus of claim 1, wherein the at least one SOI icon includes an SOI icon representing an SOI including a lesion, and
wherein the SOI icon includes an indication that the lesion is present in the SOI corresponding to the SOI icon.

10. The medical imaging apparatus of claim 1, wherein, based on receiving an input of selecting an SOI icon from among the at least one SOI icon, the processor is further configured to execute the one or more instructions to control the display to display an image showing an SOI corresponding to the selected SOI icon.

11. A method of controlling a medical imaging apparatus, the method comprising:
acquiring a rendered three-dimensional medical image of a region of interest (ROI) based on medical imaging data;
recognizing at least one structure of interest (SOI) included in the rendered three-dimensional medical image;
acquiring at least one SOI icon including at least one image showing the at least one SOI; and
displaying the at least one SOI icon.

12. The method of claim 11, wherein the at least one image includes an image obtained by reducing a portion corresponding to the at least one SOI in the rendered three-dimensional medical image or an image including a figure representing the at least one SOI that is acquired on a result of segmenting the at least one SOI.

13. The method of claim 11, wherein the acquiring of the at least one SOI icon comprises acquiring the at least one SOI icon in response to an input for acquiring the rendered three-dimensional medical image.

14. The method of claim 11, further comprising:
based on receiving an input for changing a first slice of the rendered three-dimensional medical image to a second slice, updating the rendered three-dimensional medical image of the ROI based on the medical imaging data and the second slice;
recognizing at least one new SOI included in a result of the updating; and
updating the at least one image to show the at least one new SOI.

15. The method of claim 11, further comprising:
based on receiving input for changing a first view of the rendered three-dimensional medical image to a second view, updating the rendered three-dimensional medical image of the ROI based on the medical imaging data and the second view;
recognizing at least one new SOI included in a result of the updating; and
updating the at least one image to show the at least one new SOI.

16. The method of claim 11, further comprising:
based on receiving an input for zooming the rendered three-dimensional medical image, updating the rendered three-dimensional medical image of the ROI based on the medical imaging data and the input;
recognizing at least one new SOI included in a result of the updating; and
updating the at least one image to show the at least one new SOI.

17. The method of claim 11, further comprising:
acquiring a plurality of rendered three-dimensional medical images respectively corresponding to a plurality of views; and
acquiring the at least one SOI icon corresponding to an image selected by a user from among the plurality of rendered three-dimensional medical images.

18. The method of claim 11, further comprising acquiring the medical imaging data from the medical imaging apparatus.

19. The method of claim 11, wherein the at least one SOI icon includes an SOI icon representing an SOI including a lesion, and
wherein the SOI icon includes an indication that the lesion is present in the SOI corresponding to the SOI icon.

20. A non-transitory computer-readable medium configured to store instructions which, when executed by a processor, cause the processor to perform the method of claim 11.
